(19) United States
(12) Patent Application Publication
(10) Pub. No.: US 2020/0276099 A1
(43) Pub. Date: Sep. 3, 2020

(54) PLANT-BASED COSMETIC COMPOSITIONS

(71) Applicant: L'Oreal, Clark, NJ (US)

(72) Inventors: Kyle ROBBINS, Toms River, NJ (US); Heather LEE, Wayne, NJ (US)

(21) Appl. No.: 16/796,865

(22) Filed: Feb. 20, 2020

Related U.S. Application Data
(60) Provisional application No. 62/812,230, filed on Feb. 28, 2019.

(51) Int. Cl. A61K 8/46; A61K 8/34; A61K 8/44; A61K 8/73; A61Q 5/02

(52) U.S. Cl. CPC A61K 8/463; A61K 8/342; A61K 8/345; A61K 8/44; A61K 2800/596; A61Q 5/02; A61K 2800/48; A61K 2800/30; A61K 8/737

(57) ABSTRACT

The application relates to compositions and methods for cosmetically treating and/or cleansing keratinous materials. The compositions comprise an anionic surfactant system; a nonionic surfactant system comprising one or more alkylpolyglucosides and one or more monounsaturated glyceryl esters; at least one amphoteric surfactant; and at least one thickening agent chosen from plant-based cationic polymers.

This application claims priority to U.S. Provisional Patent Application No. 62/812,230, filed Feb. 28, 2019.

The disclosure relates to natural, plant-based cosmetic compositions, such as shampoos, body cleansers, and face cleansers, for cosmetically treating, shampooing and/or cleansing keratinous materials, and to methods of cleansing keratinous materials.

### TECHNICAL FIELD

### Background

Consumers desire natural compositions for cleansing keratinous materials, including hair and skin. However, such compositions can be expensive to produce as their materials must be sourced from natural sources such as plants, as opposed to being high-volume, industrially-produced chemicals. Moreover, it is difficult to achieve an acceptable balance of desirable cleansing composition performance properties, especially when manufacturers are limited to using naturally-sourced products. For example, consumers may seek cleansing compositions that foam and cleanse well, have a certain “thickness” (viscosity), are mild to the skin and hair, and rinse away from the body with ease. However, the addition of a particular component to a cleansing composition will often enhance one desired property to the detriment of another desired property.

It is particularly difficult to replace the high-volume, low-cost industrially-produced surfactants widely used in aqueous based personal care products, with naturally-derived surfactants. From the consumer's perspective, the amount and stability of the foam directly relates to the perceived cleaning efficiency of the composition. Generally speaking, the larger the volume of foam produced and the more stable the foam, the more efficient is the perceived cleaning action of the composition. This presents a potential problem in low-surfactant formulations, as foam volume tends to decrease with decreasing surfactant concentration.

Non-natural sulfate-based surfactants (such as, for example, sodium lauryl sulfate and sodium lauryl ether sulfate) may be chosen because of their effectiveness in cleansing, foam production, and stability. Personal care cleansers containing sulfate-based surfactants are also generally easy to thicken with typical thickeners. Nonetheless, these particular surfactants can be harsh and irritating to skin. For instance, over-use of sulfate-based surfactants can cause needless drying to the face and scalp, and contribute to color fading and drying of hair.

Replacing these surfactants with natural surfactants has been difficult. The industrial process of ethoxylation makes surfactants more water soluble, and thus improves surfactant cleaning, emulsifying, and wetting properties. Yet consumers desire natural cleansing products which are equivalent in performance to the formulations that they use. Cleansing compositions are expected to adequately cleanse the hair and/or skin. Moreover, consumers desire cleansing compositions that are made up primarily of natural materials, including natural thickeners, as opposed to having just one natural ingredient. This complicates the ability to produce natural cleansing products with good cosmetic properties.

Eliminating sulfate surfactants from cleansing compositions has been particularly challenging because non-sulfate based compositions typically have poor foaming properties, are difficult to thicken, and may not provide the desired degree of clarity or transparency (if clear compositions are desired). Also, the cleansing ability of non-sulfate-based compositions may be sub-optimal.

In addition to having excellent cosmetic qualities, these natural compositions also require a certain viscosity or rheology to be recognized as a specific product—e.g., a shampoo, a body cleanser, or a face cleanser—and to be convenient for dispensing and using and thus desirable to consumers. It is known practice to use compositions that contain conventional thickeners, either natural or synthetic, in order to give such compositions a certain viscosity or rheology. Nonetheless, conventional thickeners may lack sustainable sourcing and not comply with “green” manufacturing processes, which in turn can lead to harmful effects on the environment.

Due to the concerns regarding conventional thickeners, industrial surfactants and/or sulfate-containing surfactants, manufacturers seek to formulate compositions for applying to and/or cleansing keratinous materials using ingredients and combinations of ingredients that can minimize or prevent the above-described disadvantages. However, the choice of ingredients or combinations of ingredients poses difficulties insofar as they cannot be detrimental to other cosmetic attributes such as cosmetic feel, volume, bounce, hair individualization, and suppleness; viscosity or rheology properties of cleansing compositions; stability of the compositions, or result in more disadvantages such as increased irritation of the scalp and damage to the hair. It is therefore desirable to provide the consumer with compositions that can care for and/or cleanse keratinous materials, wherein the compositions have the desired viscosity or rheology and cosmetic attributes, while being comprised of raw materials that are of sustainable sourcing and produced by green manufacturing processes. It is also desirable to provide consumers with compositions that are non-sulfate-based.

### SUMMARY

It has now been surprisingly and unexpectedly discovered that combining an anionic surfactant system comprising at least one non-ethoxylated anionic surfactant with a nonionic surfactant system, at least one amphoteric surfactant, and at least one thickening agent chosen from plant-based cationic polymers results in compositions with desired cosmetic qualities and level of structuring and viscosity (or rheology), while avoiding the use of synthetic anionic surfactants and conventional thickeners. For example, the compositions may, in various embodiments, achieve desired properties while avoid using secondary gums (e.g. non-plant-based cationic polymers such as non-guar gums), silicones, or synthetic polymers.

In various embodiments, the nonionic surfactant system comprises one or more alkylpolyglucosides and/or one or more monounsaturated glyceryl esters. In various embodiments, the nonionic surfactant system may be present in the composition in an amount ranging from about 0.1% to about 12%, by weight of the composition. In various embodiments, the alkylpolyglucosides may be present in an amount ranging from about 0.1% to about 10%, by weight of the composition. In various embodiments, the monounsaturated glyceryl esters may be present in an amount ranging from about 0.2% to about 2%, by weight of the composition.

In various exemplary embodiments, the one or more alkylpolyglucosides may be present in an amount ranging from about 3.0% to 7.5%, by weight of the composition; the one or more monounsaturated glyceryl esters may be present in an amount ranging from about 0.2% to 2.0%, by weight of the composition; and/or the weight ratio of alkyl polyglycosides to monounsaturated glyceryl esters may range from about 3:1 to about 15:1. The plant-derived natural anionic surfactant may achieve improved cleansing and foaming, and natural emollients and environmentally friendly cationic conditioning agents may achieve viscosity, suspension, and cosmeticity. The compositions may be high foaming without having to increase the amount of anionic surfactant, which can be damaging. The compositions also provide adequate foaming if formulated with non-sulfate based surfactants. Additionally, the compositions allow for the suspension of pearlizing agent without a non-natural thickener. The compositions optionally contain coco-based materials, which are desirable to consumers. Finally, the compositions are primarily made up of natural ingredients, which is a highly sought after attribute for environmentally-conscious consumers.

The disclosed embodiments provide compositions for applying to and/or cleansing keratinous materials. The disclosure relates, in various embodiments, to a cleansing composition comprising: a) an anionic surfactant system comprising at least one non-ethoxylated anionic surfactant; b) a nonionic surfactant system comprising one or more alkylpolyglucosides and one or more monounsaturated glyceryl esters; c) at least one amphoteric surfactant; and d) at least one thickening agent chosen from plant-based cationic polymers; wherein the one or more alkylpolyglucosides are present in an amount ranging from about 3.0% to 7.5%, by weight of the composition; wherein the one or more monounsaturated glyceryl esters are present in an amount ranging from about 0.2% to 2.0%, by weight of the composition; wherein the weight ratio of alkyl polyglycosides to monounsaturated glyceryl esters ranges from about 3:1 to about 15:1.

It is to be understood that both the foregoing general description and the following detailed description present various embodiments of the disclosure, and are intended to provide an overview or framework for understanding the nature and character of the claims.

### DESCRIPTION

Various aspects of the present disclosure relate to compositions comprising an anionic surfactant system comprising at least one non-ethoxylated anionic surfactant; a nonionic surfactant system comprising at least one alkylpolyglucoside and/or at least one monounsaturated glyceryl ester; at least one amphoteric surfactant; and at least one thickening agent chosen from plant-based cationic polymers.

### Anionic Surfactant System

Compositions according to the disclosure comprise an anionic surfactant system that comprises at least one ethoxylated or non-ethoxylated anionic surfactant, and may be sulfate-based or non-sulfate based. In certain embodiments, the at least one anionic surfactant may be plant-based.

The total amount of the anionic surfactant system present in the compositions according to the disclosure may vary, but is typically in the range of from about 0.1% to about 12%, such as from about 1% to about 10%, from about 2% to about 9%, about 3% to about 8%, about 3% to about 7.5%, or from about 6.2% to about 7.3%, including all ranges and sub-ranges there between, based on the total weight of the composition.

Non-limiting examples of anionic surfactants that may be used include alkyl sulfates, monoglyceride sulfates, alkylsulfonates, alkylamide sulfonates, alkylarylsulfonates, alpha-olefin sulfonates, paraffin sulfonates, alkylsulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group.

Non-limiting examples of anionic surfactants further include fatty acid soaps such as soap base, sodium laurate or sodium palmitate, alkyl sulfate ester salts having 8 to 22 carbon atoms such as sodium lauryl sulfate or potassium lauryl sulfate, N-acyl sarcosinates such as sodium lauroyl sarcosinate, fatty acid amide sulfonates having 8 to 22 carbon atoms such as sodium N-myristyl-N-methyl taurate, sodium methyl coconut oil fatty acid taurate (also referred to as sodium methyl cocoyl taurate) or sodium methyl lauryl taurate, sulfosuccinates such as sodium di(2-ethylhexyl)sulfosuccinate, sodium monolauroyl monoethanolamide polyoxyethylene sulfosuccinate or sodium lauryl polypropylene glycol sulfosuccinate, alkyl benzene sulfonates such as sodium linear dodecyl benzene sulfonate, triethanolamine linear dodecyl benzene sulfonate or linear dodecyl benzene sulfonate, N-acyl glutamates such as monosodium N-lauroyl glutamate, disodium N-stearoyl glutamate or monosodium N-myristyl-L-glutamate, N-acyl glycinates such as potassium N-cocoyl glycinate or sodium N-stearoyl glycinate, fatty acid ester sulfate ester salts having 8 to 22 carbon atoms such as sodium hydrogenated coconut oil fatty acid glyceryl sulfate, sulfonated oils such as turkey red oilα-olefin sulfonates, fatty acid ester sulfonates having 8 to 22 carbon atoms, secondary alcohol sulfate ester salts, fatty acid alkyloyl amide sulfate ester salts having 8 to 22 carbon atoms, sodium lauroyl ethanolamide succinate, ditriethanolamine N-palmitoyl aspartate and sodium casein.

When the anionic surfactants are in salt form, they may be chosen especially from alkali metal salts such as the sodium or potassium salt and preferably the sodium salt, ammonium salts, amine salts and in particular amino alcohol salts, or alkaline-earth metal salts such as the magnesium salt.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfo succinates.

In an embodiment, the anionic surfactants are selected from sodium oleyl succinate, ammonium lauryl sulfosuccinate, sodium lauryl sulfate, ammonium lauryl sulfate, sodium dodecylbenzene sulfonate, triethanolamine dodecylbenzene sulfonate, sodium cocoyl isethionate, sodium lauryl isethionate, and sodium N-lauryl sarcosinate or mixtures thereof. In a further embodiment, anionic surfactants are sodium lauryl sulfate, ammonium lauryl sulfate, or mixtures thereof.

According to at least certain embodiments, the anionic surfactants described herein have not been ethoxylated.

### Sulfate-Based Anionic Surfactants

In certain exemplary embodiments, the anionic surfactant system comprises at least one ethoxylated or non-ethoxylated anionic surfactant that is sulfate-based.

If present, the total amount of the at least one sulfate-based anionic surfactant may vary, but typically ranges from about 0.1% to about 12%, based on the total weight of the present composition. In some cases, the total amount of the sulfate-based anionic surfactant ranges from about 0.1% to about 12%, about 0.5% to about 11%, about 1 to about 10%, about 4.8% to about 6.6%, or about 3.6% to about 6.6%, including all ranges and sub-ranges there between, based on the total weight of the composition.

In an embodiment, the anionic surfactants are selected from sodium lauryl sulfate, ammonium lauryl sulfate, sodium lauroyl sarconisate, sodium oleylsuccinate, ammonium lauryl sulfosuccinate, sodium dodecylbenzol sulfonate and/or triethanolamine dodecylbenzol sulfonate or mixtures thereof.

Non-limiting examples of useful sulfate-based anionic surfactants are provided below.

### Alkyl Sulfates

In various embodiments, alkyl sulfates may be chosen as anionic surfactants. Non-limiting examples of useful alkyl sulfates include those of formula (I):

ROSO₃X⁻ (I)

wherein R represents linear or branched C₆ to C₃₀ alkyl groups, optionally derived from coconut oil; and X⁻ is a cation chosen from sodium, potassium, ammonium, substituted ammonium, or magnesium.

In certain embodiments, the at least one alkyl sulfate anionic surfactant may be chosen from C₆₋₂₄ alkyl sulfates. In certain embodiments, the alkyl sulfate anionic surfactant may be in the form of a salt having a cation chosen from sodium, potassium, ammonium, or substituted ammonium.

Non-limiting examples of useful alkyl sulfate anionic surfactants include sodium lauryl sulfate, ammonium lauryl sulfate, sodium dodecyl sulfate, sodium coco-sulfate, ammonium coco-sulfate and mixtures thereof. In certain embodiments, the alkyl sulfate anionic surfactant may be chosen from sodium lauryl sulfate. In other embodiments, the alkyl sulfate anionic surfactants are chosen from sodium coco-sulfate, ammonium coco-sulfate, or mixtures thereof.

### Non-Sulfate Based Anionic Surfactants

In further exemplary embodiments, the anionic surfactant system does not comprise sulfates, or is substantially free of sulfates. In yet further embodiments, non-sulfate based anionic surfactants are the predominant type of surfactant in the anionic surfactant system (e.g., there is a higher percentage of non-sulfate based anionic surfactants than sulfate-based anionic surfactant in the anionic surfactant system).

The total amount of non-sulfate-based anionic surfactants can vary but typically ranges from about 5 to about 50%, based on the total weight of the composition.

Non-limiting examples of non-sulfate-based anionic surfactants include alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof.

Non-limiting examples of useful non-sulfate-based anionic surfactants are provided below.

### Acyl Sarcosinates

In various embodiments, acyl sarcosinates may be chosen as anionic surfactants.

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

The total amount of acyl sarcosinates in the cleansing composition, if present, may vary but is typically from about 0.001% to about 10%, based on the total weight of the cleansing composition. In some instance, the total amount of acyl sarcosinates in the cleansing composition ranges from about 0.1% to about 6%, about 0.1% to about 4%, or about 0.1% to about 3%, including all ranges there between, based on the total weight of the composition.

### Acyl Isethionates

In various embodiments, acyl isethionates may be chosen as anionic surfactants. Non-limiting examples of useful acyl isethionates and their salts include those of formula (I) and (II):

RCOOCHR¹CHR²X⁻ M⁺ (II)

RCOOCHR¹CHR²X⁻ Na⁺ (III)

wherein:
R, R¹, and R² are each independently chosen from H or an alkyl chain having 1-24 carbon atoms, said chain being saturated or unsaturated, linear or branched;
X is COO⁻ or SO₃⁻; and
M is any suitable cation.

Although the cation may be chosen from any suitable cation as in formula (II), including, for example, alkali metal ion such as sodium or potassium, ammonium ions, or alkanolammonium ions such as monoethanolammonium or triethanolammonium ions, sodium is a preferred cation, as in formula (III). In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

The total amount of acyl isethionates in the cleansing composition, if present, may vary, but typically ranges from about 0.01% to about 10%, based on the total weight of the cleansing composition. In some instance, the total amount of acyl isethionates in the cleansing composition is from about 0.1% to about 7%, about 0.5% to about 5%, or about 0.8% to about 4.5%, including all ranges and sub-ranges there between, based on the total weight of the composition.

In some cases, more than one sulfate-containing anionic surfactant may be chosen. For example, two or more, such as three or more, sulfate-containing anionic surfactants may be selected. In other cases, more than one non-sulfate-based anionic surfactants is preferred. For example, two or more, such as three or more, non-sulfate anionic surfactants may be selected. In still other cases, a combination of sulfated and non-sulfate-based anionic surfactants may be chosen.

### Nonionic Surfactants

Compositions according to the disclosure comprise a nonionic surfactant system comprising at least two nonionic surfactants. In certain embodiments, the nonionic surfactants are plant-based.

The total amount of nonionic surfactants may vary, but typically ranges from about 0.1% to about 12%, based on the total weight of the present composition. In some cases, the total amount of the nonionic surfactants may range from about 0.5% to about 11%, about 1% to about 10%, about 3% to about 9%, about 5% to about 8%, about 6% to about 7.5%, or about 6.2% to about 7.3%, including all ranges and sub-ranges there between, based on the total weight of the composition.

In yet further embodiments, the total amount of the nonionic surfactants of the nonionic surfactant system may range from about 0.1% to about 10%, such as about 0.1% to about 9%, about 1% to about 8%, about 2% to about 8%, about 3% to about 7%, or about 3% to about 6.5%, including all ranges and sub-ranges there between, based on the total weight of the present compositions.

Non-limiting examples of useful non-ionic surfactants are included below.

### Alkyl Polyglucosides

In various embodiments, alkyl polyglucosides may be chosen as nonionic surfactants.

The total amount of alkyl polyglucosides in the cleansing compositions may vary, but typically ranges from about 0.1% to about 15%, based on the total weight of the cleansing composition. In some instance, the total amount of alkyl polyglucosides in the cleansing composition is from about 0.1% to about 10%, from about 0.5% to about 9%, from about 1% to about 8%, from about 2% to about 7%, from about 2.5% to about 6%, or from about 3% to about 5.5%, including ranges and sub-ranges there between, based on the total weight of the cleansing composition.

In yet further embodiments, the total amount of the alkyl polyglucosides may range from about 3% to about 7%, such as from about 3.5% to about 6.5%, about 4% to about 6%, or about 4.5% to about 5.5%, including all ranges and sub-ranges there between, based on the total weight of the composition.

In further embodiments still, the total amount of the alkyl polyglucosides may range from about 3% to about 12%, such as from about 4% to about 10%, about 5% to about 9%, about 5.5% to about 9%, about 6% to about 8%, or about 6% to about 7.5%, including ranges and sub-ranges there between, based on the total weight of the composition.

Exemplary polyglucosides include alkyl polyglucosides having the following formula (IV):

R¹—O—(R²O)ₙ—Z(x) (IV)

wherein:
R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Nonlimiting examples of alkyl polyglucosides include lauryl glucoside, octyl glucoside, decyl glucoside, coco glucoside, caprylyl/capryl glucoside, and sodium lauryl glucose carboxylate. In certain embodiments, the at least one alkyl poly glucoside compound is selected from the group consisting of lauryl glucoside, decyl glucoside and coco glucoside.

In certain embodiments, the at least one alkyl polyglucoside is decyl glucoside.

### Monounsaturated Glyceryl Esters

In various embodiments, monounsaturated glyceryl esters may be chosen as nonionic surfactants.

The total amount of monounsaturated glyceryl esters in the cleansing compositions may vary, but typically ranges from about 0.01% to about 10%, based on the total weight of the cleansing composition. In some instance, the total amount of monounsaturated glyceryl esters in the cleansing composition ranges from about 0.05% to about 8%, from about 0.1% to about 5%, from about 0.2% to about 2%, about 0.2% to about 1.5%, such as about 0.25% to about 1%, or from about 0.5% to about 5%, about 1% to about 3%, about 1.25% to about 2.5%, such as from about 1.5% to about 2%, including all ranges and sub-ranges there between, based on the total weight of the cleansing composition.

In further embodiments, the total amount of monounsaturated glyceryl ester may range from about 0.1% to about 5%, such as about 0.5% to about 4%, about 0.75% to about 3%, about 0.75% to about 2.25%, or about 1% to about 2%, including all ranges and sub-ranges there between, based on the total weight of the composition.

Non-limiting examples of monounsaturated glyceryl esters include glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, or mixtures thereof. In certain embodiments, the at least one monounsaturated glyceryl ester may be chosen from polyglyceryl-4 isostearate, polyglyceryl-3 oleate, polyglyceryl-2 sesquioleate, triglyceryl diisostearate, diglyceryl monooleate, tetraglyceryl monooleate, or mixtures thereof.

In certain embodiments, the at least one monounsaturated glyceryl ester may be chosen from polyglyceryl-3 caprate, polyglyceryl-4 caprate, glyceryl laurate, polyglyceryl-2 laurate, polyglyceryl-5 laurate, polyglyceryl-10 laurate, glyceryl myristate, glyceryl stearate, glyceryl undecylenate, glyceryl oleate, or mixtures thereof.

In at least one exemplary embodiment, the at least one monounsaturated glyceryl ester is chosen from glyceryl oleate.

In certain embodiments, the compositions comprise at least one alkyl polyglucoside and at least one monounsaturated glyceryl ester. In certain embodiments, the at least one alkyl polyglucosides and the at least one monounsaturated glyceryl ester are present in the compositions in a weight ratio ranging from about 1:1 to about 14:1, such as about 2:1 to about 14:1, about 2:1 to about 10:1, about 3:1 to about 8:1, or about 3:1 to about 7:1. In various embodiments, the at least one alkyl polyglucosides is decyl glucoside. In various embodiments, the at least one monounsaturated glyceryl ester is glyceryl oleate.

### Amphoteric Surfactant

Compositions according to the disclosure comprise at least one amphoteric surfactant. In certain embodiments, the amphoteric surfactants are plant-based.

The total amount of the amphoteric surfactant may vary, but typically ranges from about 0.01% to about 10%, based on the total weight of the present composition. For example, the total amount of the amphoteric surfactant may range from about 0.1% to about 8%, such as from about 0.5% to about 7%, about 0.5% to about 5%, about 1% to about 4%, about 1% to about 3%, about 1.1% to about 2.5%, about 1.3% to about 2%, or about 1.5% to about 1.8 wt. %, including all ranges and sub-ranges there between, based on the total weight of the composition.

In further embodiments, the total amount of the amphoteric surfactants may vary, but typically ranges from about 0.1% to about 5%, such as about 0.5% to about 7%, about 1% to about 5%, or about 1.5% to about 4%, including all ranges and sub-ranges there between, based on the total weight of the composition.

Non-limiting examples of amphoteric surfactants include betaines, sultaines, amphoacetates, amphoproprionates, and mixtures thereof. In certain embodiments, betaines and amphoproprionates are used. In certain embodiments, betaines are used. Betaines which can be used in the current compositions include those having the following formulas:

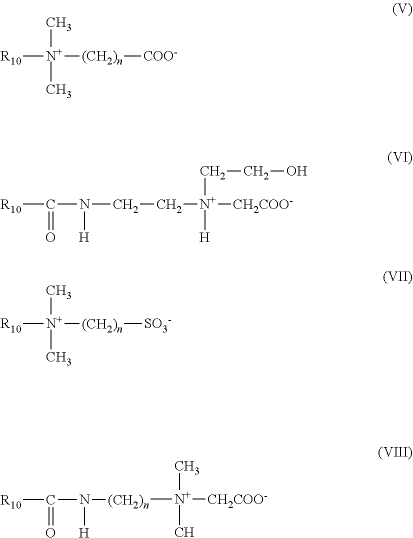

wherein:
R¹⁰ is an alkyl group having 8-18 carbon atoms; and
n is a integer from 1 to 3.

Non-limiting examples of betaines include coco betaine, cocoamidopropyl betaine, lauryl betaine, laurylhydroxy sulfobetaine, lauryldimethyl betaine, cocoamidopropyl hydroxysultaine, behenyl betaine, capryl/capramidopropyl betaine, lauryl hydroxysultaine, stearyl betaine, and mixtures thereof. Typically, the at least one betaine compound is selected from the group consisting of coco betaine, cocoamidopropyl betaine, behenyl betaine, capryl/capramidopropyl betaine, lauryl betaine, and mixtures thereof, and more typically coco betaine.

Hydroxyl sultaines useful in the compositions according to embodiments of the disclosure include the following formula (IX):

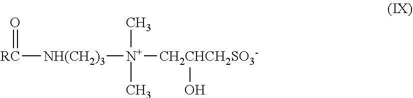

wherein:
R is an alkyl group having 8-18 carbon atoms.

Useful alkylamphoacetates include those having the formula (X):

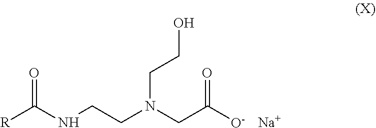

wherein:
R is an alkyl group having 8-18 carbon atoms.

Useful alkyl amphodiacetates include those having the formula (XI):

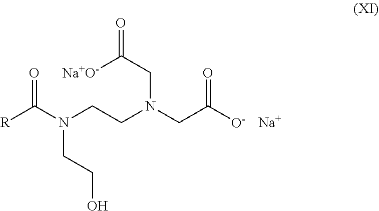

wherein:
R is an alkyl group having 8-18 carbon atoms.

The amphoteric surfactants of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, (C₈-C₂₀)alkylamido (C₁-C₆)alkylbetaines, sulfobetaines, (C₈-C₂₀)alkylsulfobetaines, (C₈-C₂₀)alkylamido(C₁-C₆)alkylsulfobetaines, (C₈-C₂₀)alkylamphoacetate, (C₈-C₂₀)alkylamphodiacetate, and mixtures thereof.

Among the optionally quaternized secondary or tertiary aliphatic amine derivatives that may be used, mention may also be made of the products of respective structures (A1) and (A2) below:

Ra—CON(Z)CH₂—(CH₂)m-N+(Rb)(Rc)(CH₂COO—) (A1)

wherein:
Ra represents a C₁₀-C₃₀ alkyl or alkenyl group derived from an acid Ra—COOH preferably present in hydrolysed coconut oil, a heptyl group, a nonyl group or an undecyl group,
Rb represents a β-hydroxyethyl group,
Rc represents a carboxymethyl group;
m is equal to 0, 1 or 2, and
Z represents a hydrogen atom or a hydroxyethyl or carboxymethyl group;

Ra′—CON(Z)CH₂—(CH₂)m′-N(B)(B′) (A2)

wherein:
B represents —CH₂CH₂OX′, with X′ representing —CH₂—COOH, CH₂—COOZ′, CH₂CH₂—COOH, —CH₂CH₂—COOZ′, or a hydrogen atom,
B′ represents —(CH₂)z-Y′, with z=1 or 2, and Y′ representing COOH, COOZ′, CH₂—CHOH—SO₃H or —CH₂—CHOH—SO₃Z′,
m′ is equal to 0, 1 or 2,
Z represents a hydrogen atom or a hydroxyethyl or carboxymethyl group,
Z′ represents an ion resulting from an alkali or alkaline-earth metal, such as sodium, potassium or magnesium; an ammonium ion; or an ion resulting from an organic amine and in particular from an amino alcohol, such as monoethanola-mine, diethanolamine and triethanolamine, monoisopropanolamine, diisopropa-nolamine or triisopropanolamine, 2-amino-2-methyl-1-propanol, 2-amino-2-methyl-1,3-propanediol and tris(hydroxymethyl)aminomethane, and
Ra′ represents a C₁₀-C₃₀ alkyl or alkenyl group of an acid Ra′COOH preferably pre-sent in hydrolysed linseed oil or coconut oil, an alkyl group, in particular a C₁₇ alkyl group, and its iso form, or an unsaturated C₁₇ group.

Exemplary amphoteric surfactants include sodium cocoamphoacetate, sodium lauroamphoacetate, sodium caproamphoacetate and sodium capryloamphoacetate. Further exemplary amphoteric surfactants include disodium cocoamphodiacetate, disodium lauroamphodiacetate, disodium caproamphodiacetate, disodium capryloamphodiacetate, disodium cocoamphodipropionate, disodium lauroam-phodipropionate, disodium caproamphodipropionate, disodium capryloamphodi-propionate, lauroamphodipropionic acid and cocoamphodipropionic acid.

Non-limiting examples that may be mentioned include the cocoamphodiacetate sold by the company Rhodia under the trade name Miranol® C2M Concentrate, the sodium cocoamphoacetate sold under the trade name Miranol Ultra C 32 and the product sold by the company Chimex under the trade name CHIMEXANE HA.

Use may also be made of the compounds of formula (XII):

Ra″—NH—CH(Y″)—(CH₂)n-C(O)—NH—(CH₂)n′-N(Rd)(Re) (XII)

wherein:
Ra″ represents a C10-C30 alkyl or alkenyl group of an acid Ra″—C(O)OH preferably present in hydrolysed linseed oil or coconut oil;
Y″ represents the group —C(O)OH, —C(O)OZ″, —CH₂—CH(OH)—SO₃H or the group CH₂—CH(OH)—SO₃—Z″, with Z″ representing a cationic counterion resulting from an alkali metal or alkaline-earth metal, such as sodium, an ammonium ion or an ion resulting from an organic amine;
Rd and Re represent, independently of each other, a C₁-C₄ alkyl or hydroxyalkyl radical; and
n and n′ denote, independently of each other, an integer ranging from 1 to 3.

Exemplary compounds include sodium diethylaminopropylcocoaspart-amide, such as the one sold by the company Chimex under the name CHIMEXANE HB.

In certain embodiments, the amphoteric surfactants are chosen from (C₈-C₂₀)alkylbetaines, (C₈-C₂₀)alkylamido(C₁-C₆)alkylbetaines, (C₈-C₂₀)alkylamphoacetates and (C₈-C₂₀)alkylamphodiacetates, and mixtures thereof.

In certain embodiments, the at least one amphoteric surfactant is chosen from (C₈-C₂₀)alkyl betaines, (C₈-C₂₀)alkylamido (C₁-C₆)alkylbetaines, (C₈-C₂₀)alkylamphoacetate, (C₈-C₂₀)alkylamphodiacetate, and their salts, and mixtures thereof. In some cases, the at least one amphoteric surfactant is selected from coco-betaine, cocamidopropylbetaine, sodium cocoamphoacetate, disodium cocoamphodiacetate, and mixtures thereof.

### Cationic Polymers

Compositions according to the disclosed embodiments comprise at least one thickening agent chosen from plant-based cationic polymers.

The total amount of the cationic polymers may vary, but typically ranges from about 0.001% to about 10%, such as from about 0.01% to about 8%, about 0.05% to about 5%, about 0.1% to about 3%, about 0.1% to about 2%, about 0.125% to about 1%, about 0.2% to about 0.75%, or about 0.25% to about 0.5%, including ranges and sub-ranges there between, based on the total weight of the present compositions.

In one embodiment, at least one thickening agent is chosen from gums. Non-limiting examples of gums include acacia, agar, algin, alginic acid, ammonium alginate, amylopectin, calcium alginate, calcium carrageenan, carrageenan, dextrin, gelatin, gellan gum, guar gum, hydroxypropyl guar, guar hydroxypropyltrimonium chloride, hydroxypropyl guar hydroxypropyltrimonium chloride, karaya gum, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

The compositions of the present disclosure comprise one or more cationic guar compounds. Non-limiting examples of the one or more cationic guar compounds are non-cellulose cationic polysaccharides, such as guar gums containing trialkylammonium cationic groups. Suitable cationic guar gum derivatives are those given the PCPC (Personal Care Products Council, formerly CTFA, designation) of guar hydroxypropyl trimonium chloride, available commercially for example as JAGUAR C13S, which has a low degree of substitution of the cationic groups and a high viscosity. The low degree of cationic substitution leads to a cationic charge density of 0.0008. Other suitable materials include that known as JAGUAR C15, having a moderate degree of substitution and a low viscosity, JAGUAR C17 (high degree of substitution, hence cationic charge density of 0.0016, high viscosity) and JAGUAR C16 which is a hydroxypropylated cationic guar derivative containing a low level of substituent groups as well as cationic quaternary ammonium groups. JAGUAR C16 has a cationic charge density of 0.0008. Guar hydroxypropyl trimonium chloride, may also be available commercially for example as N-HANCE CG13 from the company Ashland, Also suitable is hydroxypropyl guar hydroxypropyltrimonium chloride, commercially available as JAGUAR 162, which is a high transparency, medium viscosity guar having a low degree of substitution.

Such products are sold, for example, under the trade names JAGUAR C13S, JAGUAR C1000, JAGUAR C17, JAGUAR 162, JAGUAR C14S, and JAGUAR EXCEL by the company Solvay (Rhodia).

In an embodiment, the one or more cationic guar compounds of the present disclosure are selected from hydroxypropyl guar hydroxypropyltrimonium chloride, commercially available under the tradename of JAGUAR 162, and guar hydroxypropyltrimonium chloride, commercially available under the tradename of JAGUAR C13S, both sold by Solvay (Rhodia), and a mixture thereof.

In an embodiment, the one or more cationic guar compounds of the present disclosure include hydroxypropyl guar hydroxypropyltrimonium chloride. The one or more cationic guar compounds may be employed in an amount of from about 0.1% to about 2% by weight, such as from about 0.2% to about 1.5% by weight, and further such as from about 0.3% to about 1.0% by weight, based on the total weight of the hair care composition, including increments and ranges therein there between.

The total amount of the one or more cationic guar compounds may be employed in an amount from about 0.1, 0.2, 0.3, 0.4, 0.5, 0.6, 0.7, 0.8, 0.9, and 1.0 to about 2.0 percent by weight, including increments and ranges therein there between.

In certain embodiments, the at least one thickening agent may be chosen from guar gum, hydroxypropyl guar, hydroxypropyltrimonium guar, or hydroxypropyl guar hydroxypropyltrimonium guar, or salts thereof.

In certain embodiments, the cationic guar gum is a hydroxypropyl hydroxypropyltrimonium guar or salts thereof.

### pH Adjusters

The composition may also contain pH adjusters chosen from acid and alkali pH adjusters, which are well known in the art in the cosmetic treatment of keratin fibers, such as hair. Such pH adjusters include, but are not limited to, sodium metasilicate, silicate compounds, citric acid, ascorbic acid, and carbonate compounds.

In certain embodiments, the pH adjusters may be present in an amount ranging from about 0.01% to about 3% by weight, or from about 0.1% to about 2% by weight, relative to the total weight of the composition.

The pH of the compositions according to the disclosure generally ranges from about 4 to about 7, for example from about 5 to about 6.5, or from about 5 to about 6.0, or from about 5 to about 5.5, including ranges and subranges there between. In certain embodiments, the pH of the compositions according to the disclosure is at about 5, 5.1, 5.2, 5.3, 5.4, 5.5, 5.6, 5.7, 5.8, 5.9, and 6.

In certain embodiments, the pH of the composition may range from about 4 to about 9. In certain embodiments, the pH of the composition may range from about 6 to about 7.

### Cosmetically Acceptable Solvents

The compositions according to the disclosed embodiments comprise at least one cosmetically acceptable solvent. In certain embodiments, the cosmetically acceptable solvents may be chosen from organic solvents, water-soluble solvents, water, or mixtures thereof.

The total amount of cosmetically acceptable solvent in the present compositions may vary, but is typically from about 65 to about 95%, based on the total weight of the present compositions. In some cases, the total amount of water is about 70% to about 90%, about 70% to about 85%, or about 70% to about 80%, including ranges and sub-ranges there between, based on the total weight of the composition.

### Additional Ingredients

The composition according to the disclosure may also comprise additives chosen from nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preserving agents, and mixtures thereof. A person skilled in the art will take care to select the optional additives and the amount thereof such that they do not harm the properties of the compositions of the present disclosure.

The compositions of certain embodiments may comprise stabilizers, for example sodium chloride, magnesium dichloride or magnesium sulfate.

The compositions according to the disclosure may additionally comprise cosmetic adjuvants chosen from fragrances, pigments, chelating agents, softeners, antioxidants, opacifiers, stabilizers, moisturizing agents, vitamins, bactericides, preservatives, polymers, thickening agents, or any other ingredient commonly used in cosmetics for this type of application.

Preferably these additional ingredients are naturally-sourced such as from plants.

In present in the composition, these additives are generally present in an amount ranging up to about 40% by weight of active material relative to the total weight of the composition, such as up to about 30%, up to about 20%, up to about 15%, up to about 10%, up to about 5%, such as from 0% to 20%.

In certain embodiments, the compositions are substantially free of non-plant based non-cationic polymers (such as non-guar gums), silicones or synthetic polymers.

In certain embodiments, the compositions do not comprise, or are substantially free of, a pearlizing agent. In certain embodiments, the compositions comprise a pearlizing agent.

In certain embodiments, at least 75%, such as at least 80%, at least 85%, at least 90%, or at least 95% of the materials used in the composition are plant-based.

The amounts of these various constituents which can be present in the composition according to the disclosure are those conventionally used in the art. The composition according to the disclosure especially finds a particularly advantageous application in the hair sector, especially for caring for, and/or cleansing the hair or the scalp. The hair compositions are preferably shampoos, hair conditioners, styling or care gels, care lotions or creams, conditioners, masks, sera, lotions or shampoos for combating hair loss, antiparasitic shampoos, antidandruff lotions or shampoos, or shampoos for treating seborrhoea. Preferably, the composition according to the disclosure is a shampoo.

The composition according to the disclosure may be contained in a tube, in a bottle optionally equipped with a pump, or alternatively in an aerosol. In the case of an aerosol, the composition may then contain one or more standard propellants.

Advantageously, the composition according to the disclosure is in the form of a hair composition for cleansing the hair, such as a shampoo.

### Processes/Methods

The compositions according to the disclosure may be prepared according to techniques that are well known to those skilled in the art.

A subject of the disclosure is also a cosmetic treatment process or method, especially for caring for, washing/cleansing and/or conditioning keratin materials, especially the hair and/or the scalp, comprising the application to the said materials of a composition according to the disclosure.

It is in particular a cosmetic hair treatment process or method, for caring for, cosmetically treating and/or cleansing the hair and/or the scalp and/or the skin.

Embodiments of the disclosure also relate to a process for cleansing keratinous materials, such as hair, which consists in applying an effective amount of a composition as defined above to the said keratinous materials, and in rinsing, for example with water, after an optional leave-on time.

Certain embodiments also relate to a process for cleansing keratinous materials, which consists in applying an effective amount of a composition as defined above to the said keratinous materials, and in optionally rinsing, for example with water, after an optional leave-on time.

In some embodiments, keratinous materials, such as hair, may be washed or cleansed by a first step of applying the composition of the disclosure onto hair, with an optional leave-on time, followed by a second step of applying a conditioning composition, with an optional step of rinsing the hair with water between the two first and second steps, and optionally rinsing the conditioning composition, for example with water, after an optional leave-on time.

The compositions may be applied to keratinous substrates, such as the hair, and subsequently rinsed off. In various embodiments, the compositions comprise shampoo compositions for shampooing and/or conditioning the hair, and in various embodiments the shampoo composition will traditionally be rinsed off the hair within a short period of time after application to the hair, such as a period of time up to about 10 minutes, up to about 5 minutes, or up to about 2 minutes after application to the hair.

In various embodiments, processes according to the disclosure comprise applying the compositions described onto keratinous substrates, such as the hair, and subsequently rinsing the compositions off. The processes may, in various embodiments, impart conditioning and manageability to the keratinous substrate to which the composition is applied, even after the composition is rinsed off. The processes may additionally impart long lasting conditioning and manageability to the keratinous substrates.

As used herein, the method/process and composition disclosed herein may be used on the hair that has not been artificially dyed, pigmented or permed.

As used herein, the terms “comprising,” “having,” and “including” are used in their open, non-limiting sense.

The terms “a,” “an,” and “the” are understood to encompass the plural as well as the singular.

The expression “one or more” means “at least one” and thus includes individual components as well as mixtures/combinations.

Other than in the operating examples, or where otherwise indicated, all numbers expressing quantities of ingredients and/or reaction conditions are to be understood as being modified in all instances by the term “about,” meaning within +/−5%, 4%, 3%, 2%, or 1% of the indicated number. Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All percentages, parts and ratios herein are based upon the total weight of the compositions of the present disclosure, unless otherwise indicated.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

The term “substantially free” or “essentially free” as used herein means that there is less than about 5% by weight of a specific material added to a composition, based on the total weight of the compositions. The compositions of the disclosure may be free of the components or may be “substantially free” or “essentially free” of the components described for optional inclusion in said compositions. Nonetheless, the compositions may include less than about 4%, less than about 3%, less than about 2%, less about 1%, less than about 0.5%, less than about 0.1%, less than about 0.01%, less than about 0.001%, or none of the specified material or components. Likewise, as used herein, the term “avoid” or “avoiding” the inclusion of a component means that there is less than about 5% by weight of a specific material added to a composition, based on the total weight of the compositions, such as less than about 4%, less than about 3%, less than about 2%, less about 1%, less than about 0.5%, less than about 0.1%, less than about 0.01%, less than about 0.001%, or none of the specified material or components.

The term “surfactant system” refers to one surfactant or a combination of different surfactants. For example, the term “anionic surfactant system” refers to one anionic surfactant or a combination of different anionic surfactants, and the term “nonionic surfactant system” refers to one nonionic surfactant or a combination of different nonionic surfactants.

The term “synthetic” means a material that is not of natural origin. The term “natural” and “naturally-sourced” means a material of natural origin, such as derived from plants, which also cannot be subsequently chemically or physically modified. Plant-based” means that the material came from a plant.

“Cosmetically treated” means treated to improve or restore appearance.

“Non-sulfate-based” or “non-sulfate” anionic surfactants means that the surfactant does not comprise a sulfate group.

Unless otherwise expressly stated, it is in no way intended that any method set forth herein be construed as requiring that its steps be performed in a specific order. Accordingly, where a method claim does not expressly recite an order to be followed by its steps or it is not otherwise specifically stated in the claims or descriptions that the steps are to be limited to a specific order, it is no way intended that any particular order be inferred.

### EXAMPLES

The following examples serve to illustrate the embodiments of the disclosure without however exhibiting a limiting character. In these examples the amounts of the composition ingredients are given as weight percentages of active ingredients relative to the total weight of the composition.

### Example 1

Formulations comprising the following ingredients were prepared and are disclosed in Tables 1-3 below. Amounts are expressed in wt % of active materials:

TABLE 1
Formulations with Non-Ethoxylated Sulfate-Based Anionic Surfactants

| US INCI NAME | Composition A | Composition B | Composition C | Composition D | Composition E |
|---|---|---|---|---|---|
| GLYCOL DISTEARATE | 0.50 | 0.50 | 0 | 0 | 0 |
| NON-ETHOXYLATED | 5.90 | 5.90 | 5.90 | 5.32 | 5.90 |
| ANIONIC SURFACTANT | | | | | |
| SODIUM LAURYL | | | | | |
| SULFATE | | | | | |
| COCO-GLUCOSIDE | 3.52 | 3.52 | 3.52 | 3.52 | 3.52 |
| SODIUM BENZOATE | 0.5 | 0.5 | 0.5 | 0.5 | 0.5 |
| GLYCERYL OLEATE | 0.53 | 0.53 | 0.53 | 1.06 | 0.53 |
| COCO-BETAINE | 1.6 | 1.6 | 1.7 | 1.67 | 1.67 |
| SALICYLIC ACID | 0.20 | 0.20 | 0.2 | 0.2 | 0.2 |
| HYDROXYPROPYL GUAR | 0.4 | 0.4 | 0.4 | 0.17 | 0.4 |
| HYDROXYPROPYLTRIMONIUM | | | | | |
| CHLORIDE | | | | | |
| SODIUM CHLORIDE | 1.35 | 1.35 | 1.36 | 1.36 | 1.36 |
| WATER | Q.S. 100 | Q.S. 100 | Q.S. 100 | Q.S. 100 | Q.S. 100 |
| TRIETHYL CITRATE | 0.5 | 0.5 | 0.5 | 0 | 0 |
| GLYCERIN | 0 | 0.05 | 0 | 0 | 0 |

TABLE 2
Formulations with Non-Ethoxylated Sulfate-Based Anionic Surfactants

| US INCI NAME | Composition F | Composition G | Composition H | Composition I |
|---|---|---|---|---|
| NON-ETHOXYLATED | 6.24 | 5.90 | 6.24 | 6.24 |
| ANIONIC SURFACTANT | | | | |
| SODIUM COCO-SULFATE | | | | |
| GLYCOL DISTEARATE | 0 | 0 | 1.5 | 1.5 |
| COCO-GLUCOSIDE | 3.52 | 3.52 | 3.52 | 3.52 |
| SODIUM BENZOATE | 0.5 | 0.5 | 0.5 | 0.5 |
| GLYCERYL OLEATE | 0.53 | 0.53 | 0.53 | 0.26 |
| COCO-BETAINE | 1.47 | 1.47 | 1.6 | 1.5 |
| SALICYLIC ACID | 0.2 | 0.2 | 0.2 | 0.2 |
| HYDROXYPROPYL GUAR | 0.4 | 0.4 | 0.4 | 0.4 |
| HYDROXYPROPYLTRIMONIUM | | | | |
| CHLORIDE | | | | |
| SODIUM CHLORIDE | 1.32 | 1.32 | 1.35 | 1.33 |
| WATER | Q.S. 100 | Q.S. 100 | Q.S. 100 | Q.S. 100 |
| TRIETHYL CITRATE | 0.5 | 0.5 | 0.5 | 0.5 |
| NON-ETHOXYLATED | 0 | 18.34 | 0 | 0 |
| ANIONIC SURFACTANT | | | | |
| SODIUM LAURYL SULFATE | | | | |

TABLE 3
Formulations with Non-Ethoxylated Non-
Sulfate-Based Anionic Surfactants

| | Compo- | Compo- | Compo- |
|---|---|---|---|
| US INCI NAME | sition J | sition K | sition L |
| NON-ETHOXYLATED, | 3 | 3 | 3 |
| NON-SULFATE-BASED | | | |
| ANIONIC SURFACTANT | | | |
| SODIUM LAUROYL | | | |
| SARCOSINATE | | | |
| SODIUM BENZOATE | 0.52 | 0.52 | 0.52 |
| DECYL GLUCOSIDE | 7.42 | 7.42 | 7.42 |
| NON-ETHOXYLATED | 1.75 | 5 | 5 |
| ANIONIC SURFACTANT | | | |
| SODIUM COCOYL | | | |
| ISETHIONATE | | | |
| GLYCERYL OLEATE | 2.0 | 2.0 | 2.0 |
| COCO-BETAINE | 2.7 | 2.7 | 2.7 |
| SALICYLIC ACID | 0.2 | 0.2 | 0.2 |
| HYDROXYPROPYL GUAR | 0.65 | 0.5 | 0.5 |
| HYDROXYPROPYLTRIMONIUM | | | |
| CHLORIDE | | | |
| SODIUM CHLORIDE | 0.59 | 0.59 | 0.59 |
| WATER | Q.S. 100 | Q.S. 100 | Q.S. 100 |

### Example 2

The following compositions in Table 4 were prepared. Amounts are expressed in wt % of active materials:

TABLE 4
Shampoos with a Non-Ethoxylated Sulfate-Based Anionic Surfactant
vs. an Ethoxylated Sulfate-Based Anionic Surfactant

| | Non-Ethoxylated | Ethoxylated |
|---|---|---|
| | Surfactant | Surfactant |
| US INCI NAME | Shampoo | Shampoo |
| NON-ETHOXYLATED | 5.90 | 0 |
| ANIONIC SURFACTANT | | |
| SODIUM LAURYL | | |
| SULFATE | | |
| ETHOXYLATED | 0 | 8.4 |
| ANIONIC SURFACTANT | | |
| SODIUM LAURETH | | |
| SULFATE | | |
| COCO-GLUCOSIDE | 3.52 | 0 |
| SODIUM BENZOATE | 0.5 | 0.5 |
| GLYCERYL OLEATE | 0.53 | 0 |
| COCO-BETAINE | 1.69 | 3 |
| SALICYLIC ACID | 0.2 | 0.2 |
| HYDROXYPROPYL GUAR | 0.4 | 0 |
| HYDROXYPROPYLTRIMONIUM | | |
| CHLORIDE | | |
| SODIUM CHLORIDE | 1.36 | 1.75 |
| WATER | Q.S. 100 | Q.S. 100 |
| HEXYLENE GLYCOL | 0 | 0.6 |
| GLYCERIN | 0 | 2 |
| PEG-60 HYDROGENATED | 0 | 0.9 |
| CASTOR OIL | | |
| POLYQUATERNIUM-10 | 0 | 0.6 |

A shampoo with a non-ethoxylated sulfate-based anionic surfactant according to Example 2 (“Non-Ethoxylated Surfactant Shampoo”) was tested and compared to a shampoo with an ethoxylated sulfate-based anionic surfactant (“Ethoxylated Surfactant Shampoo”) on 10 volunteers by applying Non-Ethoxylated Surfactant Shampoo to hair on half of the volunteer's head and Ethoxylated Surfactant Shampoo on the other half of the head, lathering, rinsing, and then blow drying the hair. Non-Ethoxylated Surfactant Shampoo provided improved cosmetic properties to hair over Ethoxylated Surfactant Shampoo. Wet hair cleansed with Non-Ethoxylated Surfactant Shampoo was easier to comb through, easier to pass fingers through, easier to detangle, and easier to blow dry. Once dry, hair cleansed with Non-Ethoxylated Surfactant Shampoo has less dry ends (look/feel).

### Example 3

TABLE 5
Shampoos with a Non-Ethoxylated Sulfate-Based Anionic Surfactant
vs. an Ethoxylated Sulfate-Based Anionic Surfactant

| | Non-Ethoxylated | Ethoxylated |
|---|---|---|
| | Surfactant | Surfactant |
| US INCI NAME | Shampoo | Shampoo |
| NON-ETHOXYLATED SULFATE- | 5.32 | 0 |
| BASED ANIONIC SURFACTANT | | |
| SODIUM LAURYL SULFATE | | |
| ETHOXYLATED SULFATE- | 0 | 13.86 |
| BASED ANIONIC SURFACTANT | | |
| SODIUM LAURETH SULFATE | | |
| GLYCOL DISTEARATE | 0 | 0.129 |
| SILICONEs: | 0 | 1.5 to 2.0 |
| POLYDIMETHYLSILOXANE | | |
| AND/OR AMINO SILICONE | | |
| NONIONIC SURFACTANTS | | <0.5 |
| COCO-GLUCOSIDE | 3.52 | 0 |
| PEG-45M | 0 | 0.01 |
| COCAMIDOPROPYL BETAINE | 0 | 1.48 |
| COCO-BETAINE | 1.7 | <0.1 |
| GUAR | 0 | <0.5 |
| HYDROXYPROPYLTRIMONIUM | | |
| CHLORIDE | | |
| HYDROXYPROPYL GUAR | 0.4 | 0 |
| HYDROXYPROPYLTRIMONIUM | | |
| CHLORIDE | | |
| GLYCERYL OLEATE | 1.0 | 0 |
| SALICYLIC ACID | 0.2 | 0.2 |
| CARBOMER | 0 | <0.5 |
| SODIUM CHLORIDE | 1.4 | 1.0 |
| ORGANIC SOLVENTS: | <1.0 | <1.0 |
| GLYCERIN AND/OR | | |
| HEXYLENE GLYCOL | | |
| WATER | Q.S. 100 | Q.S. 100 |

A shampoo with a non-ethoxylated sulfate-based anionic surfactant according to Example 3 (“Non-Ethoxylated Surfactant Shampoo”) was tested and compared to a shampoo with an ethoxylated sulfate-based anionic surfactant (“Ethoxylated Surfactant Shampoo”) on 10 volunteers by applying Non-Ethoxylated Surfactant Shampoo to hair on half of the volunteer's head and Ethoxylated Surfactant Shampoo on the other half of the head, lathering, rinsing, and then blow drying the hair. Non-Ethoxylated Surfactant Shampoo provided improved cosmetic properties to hair over Ethoxylated Surfactant Shampoo. Once dry, hair cleansed with Non-Ethoxylated Surfactant Shampoo exhibited more volume.

1.-66. (canceled)

67. A cleansing composition comprising:
a) an anionic surfactant system comprising at least one non-ethoxylated anionic surfactant;
b) a nonionic surfactant system comprising at least one alkylpolyglucoside and at least one monounsaturated glyceryl ester;
c) at least one amphoteric surfactant; and
d) at least one thickening agent chosen from plant-based cationic polymers;
wherein the at least one alkylpolyglucoside is (are) present in an amount ranging from about 3.0% to 7.5%, by weight of the composition;
wherein the at least one monounsaturated glyceryl ester is (are) present in an amount ranging from about 0.2% to 2.0%, by weight of the composition;
wherein the weight ratio of alkyl polyglycoside(s) to monounsaturated glyceryl ester(s) ranges from about 3:1 to about 15:1.

68. The cleansing composition of claim 67, wherein the non-ethoxylated anionic surfactant comprises at least one sulfate-based non-ethoxylated anionic surfactant.

69. The cleansing composition of claim 68, wherein the at least one sulfate-based non-ethoxylated anionic surfactant is (are) present in the composition in an amount ranging from about 0.1% to about 12%, by weight of the composition.

70. The cleansing composition of claim 68, wherein the at least one sulfate-based non-ethoxylated anionic surfactant comprises at least one alkyl sulfate, and wherein the at least one alkyl sulfate anionic surfactant is (are) chosen from sodium lauryl sulfate, ammonium lauryl sulfate, sodium coco-sulfate, ammonium coco-sulfate, or a mixture thereof.

71. The cleansing composition of claim 68, wherein the at least one alkylpolyglucoside is (are) chosen from those having the following formula:
R¹—O—(R²O)ₙ—Z(ₓ)
wherein:
R¹ is an alkyl group having 8-18 carbon atoms;
R² is an ethylene or propylene group;
Z is a saccharide group with 5-6 carbon atoms;
n is an integer ranging from 0 to 10; and
x is an integer ranging from 1 to 5.

72. The cleansing composition of claim 68, wherein the at least one alkylpolyglucoside is (are) chosen from lauryl glucoside, octyl glucoside, decyl glucoside, coco glucoside, caprylyl/capryl glucoside, sodium lauryl glucose carboxylate, or mixtures thereof.

73. The cleansing composition of claim 68, wherein the at least one alkylpolyglucoside is (are) present in an amount ranging from about 3% to about 5.5%, by weight of the composition.

74. The cleansing composition of claim 68, wherein the at least one monounsaturated glyceryl ester comprises glyceryl oleate.

75. The cleansing composition of claim 68, wherein the at least one monounsaturated glyceryl ester is (are) present in an amount ranging from about 0.2% to about 1%, by weight of the composition.

76. The cleansing composition of claim 68, wherein the weight ratio of alkyl polyglycoside is (are) to glyceryl oleate ranges from about 3:1 to about 7:1.

77. The cleansing composition of claim 68, wherein the at least one amphoteric surfactant is (are) present in an amount ranging from about 0.1% to about 10%, by weight of the composition.

78. The cleansing composition of claim 68, wherein the at least one amphoteric surfactant comprises a betaine.

79. The cleansing composition of claim 68, wherein the at least one plant-based cationic polymer is (are) present in an amount ranging from about 0.1% to about 0.7%, by weight of the composition.

80. The cleansing composition of claim 68, wherein the at least one thickening agent chosen from plant-based cationic polymers comprises a cationic guar gum.

81. The cleansing composition of claim 80, wherein the at least one cationic guar gum is (are) a hydroxypropyl hydroxypropyltrimonium guar or salts thereof.

82. The cleansing composition of claim 67, wherein the at least one non-ethoxylated anionic surfactant system comprises at least one non-sulfate-based non-ethoxylated anionic surfactant.

83. The cleansing composition of claim 82, wherein the at least one non-sulfate-based non-ethoxylated anionic surfactant comprises at least one sarcosinate.

84. The cleansing composition of claim 82, wherein the composition is substantially free of sulfates.

85. A shampoo composition comprising: composition (1) or composition (2), wherein composition (1) comprises:
a) an anionic surfactant system comprising at least one alkyl sulfate, wherein said alkyl sulfate is (are) present in an amount ranging from about 3.6 to about 6.6%, by weight of the composition;
b) a nonionic surfactant system comprising at least one alkylpolyglucoside and at least one monounsaturated glyceryl ester, wherein the at least one alkylpolyglucoside is (are) present in an amount ranging from about 3.0% to about 7.5%, by weight of the composition; wherein the at least one monounsaturated glyceryl ester is (are) present in an amount ranging from about 0.2% to about 2.0%, by weight of the composition; wherein the weight ratio of alkyl polyglycoside(s) to monounsaturated glyceryl ester(s) ranges from about 3:1 to about 15:1;
c) at least one amphoteric surfactant, wherein the amphoteric surfactant is (are) present in an amount ranging from about 1% to about 3.9%, by weight of the composition; and
d) at least one thickening agent chosen from plant-based cationic polymers wherein the amphoteric surfactant is (are) present in an amount ranging from about 0.1% to about 0.7%, by weight of the composition,
wherein composition (2) comprises:
a) an anionic surfactant system comprising at least one sarcosinate type surfactants and at least one alkyl isethionate type surfactant, wherein said sarcosinate type surfactant is (are) present in an amount ranging up to about 3%, by weight of the composition, wherein said sarcosinate alkyl isethionate type surfactant is (are) present in an amount ranging from about 0.8 to about to about 4.5%, by weight of the composition;
b) a nonionic surfactant system comprising at least one alkylpolyglucoside and at least one monounsaturated glyceryl ester, wherein the at least one alkylpolyglucoside is (are) present in an amount ranging from about 3.0% to about 7.5%, by weight of the composition; wherein the at least one monounsaturated glyceryl ester is (are) present in an amount ranging from about 0.2% to about 2.0%, by weight of the composition; wherein the weight ratio of alkyl polyglycoside(s) to monounsaturated glyceryl ester(s) ranges from about 3:1 to about 15:1;
c) at least one amphoteric surfactant, wherein the amphoteric surfactant is (are) present in an amount ranging from about 1% to about 3.9%, by weight of the composition; and
d) at least one thickening agent chosen from plant-based cationic polymers wherein the amphoteric surfactant is (are) present in an amount ranging from about 0.1% to about 0.7%, by weight of the composition.

86. A method of cleansing hair, said method comprising:
(i) applying to the hair a composition comprising:
a) an anionic surfactant system comprising at least one non-ethoxylated anionic surfactant;
b) a nonionic surfactant system comprising at least one alkylpolyglucoside and at least one monounsaturated glyceryl ester;
c) at least one amphoteric surfactant; and
d) at least one thickening agent chosen from plant-based cationic polymers;
wherein the at least one alkylpolyglucoside is (are) present in an amount ranging from about 3.0% to 7.5%, by weight of the composition;
wherein the at least one monounsaturated glyceryl ester is (are) present in an amount ranging from about 0.2% to 2.0%, by weight of the composition;
wherein the weight ratio of alkyl polyglycoside(s) to monounsaturated glyceryl ester(s) ranges from about 3:1 to about 15:1.
(ii) rinsing the hair.
